(19) United States
(12) Patent Application Publication
(10) Pub. No.: US 2020/0243196 A1
(43) Pub. Date: Jul. 30, 2020

(54) BIOLOGICAL INFORMATION PROCESSING SYSTEM, BIOLOGICAL INFORMATION PROCESSING METHOD, AND BIOLOGICAL INFORMATION PROCESSING PROGRAM RECORDING MEDIUM

(71) Applicant: NEC CORPORATION, Tokyo (JP)

(72) Inventors: Yuji OHNO, Tokyo (JP); Masahiro KUBO, Tokyo (JP); Hiroaki FUKUNISHI, Tokyo (JP); Masahiro HAYASHITANI, Tokyo (JP); Yuan LUO, Tokyo (JP); Yutaka UNO, Tokyo (JP); Shigemi KITAHARA, Tokyo (JP)

(73) Assignee: NEC CORPORATION, Tokyo (JP)

(21) Appl. No.: 16/755,110

(22) PCT Filed: Oct. 5, 2018
(86) PCT No.: PCT/JP2018/037384

§ 371 Date: Apr. 9, 2020

(30) Foreign Application Priority Data
Oct. 10, 2017 (JP) 2017-196797

(51) Int. Cl. G16H 50/30; G16H 50/20; A61B 5/00

(52) U.S. Cl. CPC G16H 50/30; A61B 5/74; G16H 50/20

(57) ABSTRACT

The purpose of the present invention is to estimate response information with which it is possible to quickly relieve problems caused by agitation, the state of the patient, etc. A biological information processing system comprises: a determination unit that, on the basis of the features of biological information of a subject patient to be entered, determines discrimination information indicating whether the condition of the patient has changed in comparison with a normal state, and an estimation unit that estimates countermeasure information oriented for the patient on the basis of the discrimination information and countermeasure prediction parameters learned in advance.

### TECHNICAL FIELD

The present invention relates to a biological information processing system, a biological information processing method, and a biological information processing program recording medium.

### BACKGROUND ART

By measuring biological information of a patient, observing a state of the patient and predicting a state which could possibly occur in the patient have been carried out.

Patent Literature 1 discloses a technical idea comprising generating a condition score by measuring physiological information of a user by means of a sensor; predicting occurrence of an adverse condition for the user by comparing the condition score with a threshold value; and alerting a caregiver.

Patent Literature 2 discloses a technical idea for monitoring vital signs or non-vital signs by using automatic sensors and electronic signal processing in order to detect occurrence or recurrence of a physiological event such as a chronic disease or a disease. Specifically, the technical idea described in Patent Literature 2 comprises a control device which determines a level of an agitation state of a test subject in response to a sensed motion and generates, in response thereto, an alert to a clinician in order to assign a turn protocol to the test subject.

Patent Literature 3 discloses a technical idea of proposing, by learning past physiological findings of a patient, past treatments, and associated past clinical scores, an optimum treatment method when the patient encounters a condition similar to past invasion and treatment.

Patent Literature 4 discloses a technical idea comprising measuring biological information of a user such as blood pressure or body temperature; and comparing the measured biological information with a determination value to decide whether a health condition of the user is “normal”, “non-normal”, or “abnormal”.

### CITATION LIST

### Patent Literatures

PL 1: JP 5657315 B

PL 2: JP 5951630 B

PL 3: JP 2013-154190 A

PL 4: JP 2014-186402 A

### SUMMARY OF INVENTION

### Technical Problem

However, in Patent Literature 1 and Patent Literature 2, a countermeasure (measure, procedure) performed on the patient after the abnormality is detected is mostly entrusted to a responder, such as a nurse, a caregiver, and a therapist (which will be called a nurse or the like hereinafter), within a range instructed by a doctor. Therefore, an effect of the countermeasure depends on experience and intuition of the responder, chemistry between the patient and the responder, and so on. In this case, if the countermeasure is not appropriate, there are possibilities that the abnormality of the patient is hardly settled or prognosis of the patient becomes worse. Specifically, it is supposed, for example, that the responder administers a stronger sedative drug than necessary to, or imposes a strong restraint on the patient who is predicted to perform a problem behavior in the future also. In this event, although occurrence of agitation and the problem behavior associated therewith are suppressed, a load imposed on the patient is increased. In addition, since the patient tends to be confined to his/her bed for a long time, there are possibilities that recovery of the patient is delayed and the prognosis becomes worse. Furthermore, in Patent Literature 1 and Patent Literature 2, appropriate procedure may be different for every patient. Therefore, a load on the responder is increased because he/she considers the appropriate procedure for every patient. In addition, there are possibilities that an inappropriate procedure is performed on a target patient and the abnormality is not settled to cause another problem.

Patent Literature 3 proposes a recommended measure in accordance with a symptom of the patient based on past case examples. However, in Patent Literature 3, there is a problem that no consideration is made of a load on the patient due to performing the recommended measure, a load on the responder, and a surrounding environment. This is because the recommended measure is proposed based on a measured state of the patient alone. Therefore, in Patent Literature 3, there are possibilities that, on performing the recommended measure on the patient, the load on the patient becomes large and the load on the responder becomes large. In particular, in Patent Literature 3, there are possibilities that the loads on the patient and the responder become large because the recommended measure is performed after the abnormality occurs in the patient.

In Patent Literature 4, when it is determined that a condition of the user is abnormal, aid activities are assisted by displaying map information indicative of a route to the nearest hospital and an emergency contact address. However, in Patent Literature 4, there is a possibility that an aid person cannot perform an appropriate procedure in a case where the condition of the patient is urgent. This is because information including a specific countermeasure is not displayed.

It is an object of the present invention to provide a biological information processing system, a biological information processing method, and a biological information processing program recording medium, which can resolve the above-mentioned problems.

### Solution to Problem

A biological information processing system according to a first aspect of the present invention comprises a determination unit configured to determine, based on features of input biological information of a target patient, discrimination information indicating whether or not a condition of the target patient has changed in comparison with a normal state; and an estimation unit configured to estimate countermeasure information for the target patient based on the discrimination information and countermeasure prediction parameters which are preliminary learned.

A biological information processing method according to a second aspect of the present invention comprises determining, by a determination unit, based on features of input biological information of a target patient, discrimination information indicating whether or not a condition of the target patient has changed in comparison with a normal state; and estimating, by an estimation unit, countermeasure information for the target patient based on the discrimination information and countermeasure prediction parameters which are preliminarily learned.

A biological information processing program recording medium according to a third aspect of the present invention records a biological information processing program which causes a computer to execute the processes of determining, based on features of input biological information of a target patient, discrimination information indicating whether or not a condition of the target patient has changed in comparison with a normal state; and estimating countermeasure information for the target patient based on the identification information and countermeasure prediction parameters which are preliminarily learned.

### Advantageous Effect of the Invention

According to the present invention, it is possible to provide a biological information processing system, a biological information processing method, and a biological information processing program recording medium, which are capable of estimating countermeasure information that makes it possible to suppress occurrence of a non-normal condition of a target patient or that makes it possible to early settle an abnormal condition of the patient which has already occurred.

### BRIEF DESCRIPTION OF THE DRAWINGS

FIG. 1 is a block diagram for illustrating a configuration of a biological information processing system according to a first example embodiment of the present invention;

FIG. 2 is a view for illustrating an example of discrimination information according to the example embodiment of the present invention;

FIG. 3 is a view for illustrating an example of countermeasure information according to the example embodiment of the present invention;

FIG. 4 is a flow chart for illustrating an example of a flow of operation of the biological information processing system according to the first example embodiment of the present invention;

FIG. 5 is a block diagram for illustrating a configuration of a biological information processing system according to a second example embodiment of the present invention;

FIG. 6 is a block diagram for illustrating a configuration of a training unit of the biological information processing system according to the second example embodiment of the present invention;

FIG. 7 is a flow chart for illustrating an example of a flow of operation when the biological information processing system according to the second example embodiment of the present invention learns parameters;

FIG. 8A is a view for illustrating an example of data for learning countermeasure prediction parameters by the biological information processing system according to the second example embodiment of the present invention and is a view for illustrating a case where an agitation state continues;

FIG. 8B is a view for illustrating an example of data for learning the countermeasure prediction parameters by the biological information processing system according to the second example embodiment of the present invention and is a view for illustrating a case where a non-agitation state continues;

FIG. 9 is a flow chart for illustrating a flow of operation until the biological information processing system according to the second example embodiment of the present invention notifies of countermeasure information; and

FIG. 10 is a block diagram for illustrating an example of hardware configuration of the biological information processing systems according to the example embodiments of the present invention.

### DESCRIPTION OF EMBODIMENTS

Now, example embodiments of the present invention will be described in detail with reference to the drawings. Note that, in respective figures, the same or the corresponding parts are assigned with the same symbols and descriptions thereof are omitted as appropriate.

### First Example Embodiment

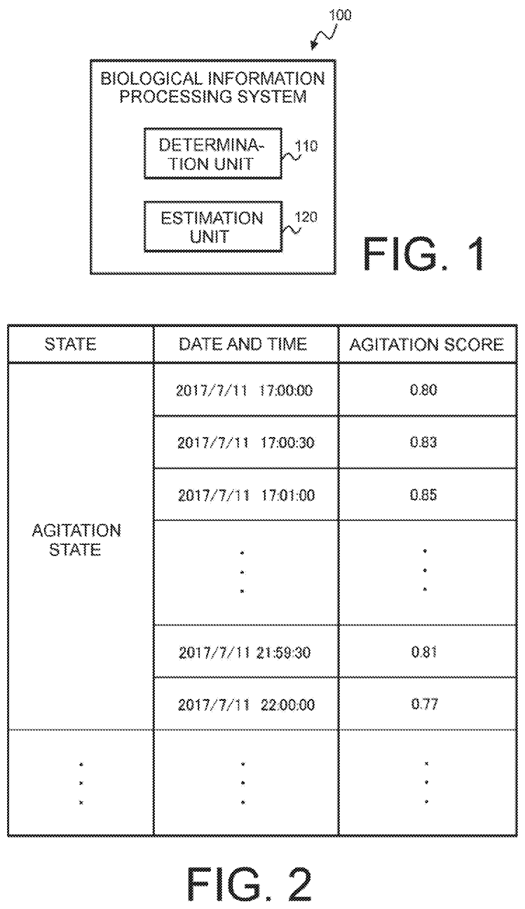

FIG. 1 is a block diagram for illustrating a configuration of a biological information processing system according to a first example embodiment of the present invention. As illustrated in FIG. 1, the biological information processing system 100 comprises a determination unit 110 and an estimation unit 120.

The determination unit 110 receives features related to biological information of a target patient and determines, based on the features, discrimination information indicating whether or not a condition of the target patient has changed in comparison with a normal state. In this example embodiment, the biological information means information related to a living body that can be measured by sensors or the like. Specifically, the biological information includes, for example, a heartbeat (pulsation), breathing, blood pressure, deep-body temperature, a level of consciousness, skin temperature, skin conductance response (Galvanic Skin Response (GSR)), a skin potential, a myoelectric potential, an electrocardiographic waveform, an electroencephalographic waveform, a sweating amount, a blood oxygen saturation level, a pulse waveform, optical brain function mapping (Near-Infrared Spectroscopy (NIRS)), a urine volume, and pupil reflex, but is not limited thereto. The features related to the biological information are information indicating characteristics of the biological information that are generated by processing the biological information of the patient and are, for example, data indicating a temporal change of the biological information in a specific frequency band. Specifically, the determination unit 110 can automatically determine, based on the features related to the biological information of the target patient, whether the target patient is in an agitation state or a non-agitation state. Herein, the agitation state (which may be called agitation hereinafter) means a state where the target patient can exhibit any problem behavior. Specifically, the agitation state includes, for example, a state where the behavior of the target patient is excessive and restless, a state where the target patient is not calm, and a state where the target patient cannot normally control his/her mind.

In addition, the problem behavior means a behavior such that he/she injures him/herself, he/she injures someone, he/she imposes a load on a nurse or the like, or an appropriate treatment cannot be continued for the patient. Specifically, the problem behavior includes, for example, a behavior such that the patient sits up on the bed, removes a fence of the bed, leaves the bed, walks by oneself, wanders around, goes to another floor in a hospital, falls down from the bed, touches a drip, a tube or the like, evulses the drip, the tube or the like, utters a strange sound, verbally abuses, or uses violence. However, a behavior corresponding to the problem behavior differs depending on a condition of the patient. The determination unit 110 may have a function of receiving the biological information of the target patient to calculate the features of the biological information. In this event, the determination unit 110 can calculate the features by carrying out leveling processing or differential processing on the biological information. The determination unit 110 may comprise, for example, a plurality of bandpass filters having different passbands, a differential filter, and so on to calculate the features (vector) by combining a plurality of values obtained by filtering processing on the biological information by using a single filter or a plurality of filters in combination.

In this example embodiment, the discrimination information is information indicating whether or not the condition of the target patient has changed in comparison with the normal state. For instance, the discrimination information means information including an agitation score indicating a possibility that the target patient is in the agitation state. For example, the agitation score is determined based on discrimination parameters which are preliminarily learned and the features related to the biological information of the target patient. Herein, the discrimination parameters mean parameters in which the features of the biological information are associated with the agitation state or the non-agitation state. For example, such discrimination parameters may be generated by machine-learning features of the biological information obtained during the agitation state and features of the biological information obtained during the non-agitation state. Such discrimination parameters may be, for example, held in a storage device (not shown) installed outside the biological information processing system 100. If the determination unit 110 comprises a storage unit (not shown), the storage unit of the determination unit 110 may hold the discrimination parameters. As described above, the discrimination parameters include the parameters in which the features of the biological information are associated with the agitation state or the non-agitation state. It is therefore possible to improve accuracy of the discrimination information by making the discrimination parameters proper.

In this example embodiment, the agitation score means an index indicating whether the target patient is in the agitation state or the non-agitation state. Specifically, the agitation score may be represented, for example, by a number which is not less than 0 and which is not more than 1. In this event, it is meant, for example, that the target patient has a high possibility of being in the agitation state or is in a strong agitation state as the agitation score is closer to 1 whereas the target patient has a high possibility of being in the non-agitation state as the agitation score is closer to 0. In addition, among numbers which are not less than 0 and are not more than 1, any number may be defined as a threshold value. In this event, the determination unit 110 may determine whether the target patient is in the agitation state or the non-agitation state depending on whether or not the agitation score of the target patient exceeds the threshold value. Furthermore, the agitation score may be represented by two values of 0 and 1. Specifically, the determination unit 110 may produce, for example, 0 when the agitation score is less than the threshold value and may produce 1 when the agitation score is not less than the threshold value. In this case, it may be determined, for example, that the target patient is in the agitation state when the agitation score is 1 whereas the target patient is in the non-agitation state when the agitation score is 0. The determination unit 110 can automatically determine the discrimination information (agitation score) of the target patient based on, for example, input features of the biological information of the target patient and the discrimination parameters which are supplied from the outside.

FIG. 2 is a view for illustrating an example of the discrimination information. As illustrated in FIG. 2, the discrimination information at least includes a state of the target patient, a date and time when the biological information is measured, and the agitation score. Specifically, the discrimination information illustrated in FIG. 2 shows, for example, that the agitation score of the target patient is “0.80” at “17:00:00 on Jul. 11, 2017,” and a state of the target patient is the “agitation state”. Although the discrimination information illustrated in FIG. 2 includes the state of the target patient and the agitation score every thirty seconds, this is an example and does not limit a measurement interval of the biological information.

The estimation unit 120 estimates, based on the discrimination information (agitation score) determined by the determination unit 110 and countermeasure prediction parameters which are preliminarily learned, countermeasure information which at least includes a countermeasure to be performed on the target patient and a countermeasure score indicating a degree of an effect of the countermeasure. Herein, the countermeasure prediction parameters mean parameters in which countermeasures performed on the target patient in the agitation state in the past are associated with a change in features related to the biological information in a predetermined period of time before and after the countermeasure is performed or with a change in agitation score. Such countermeasure prediction parameters may be generated, for example, by machine-learning the change in features of the biological information obtained by performing the countermeasure or the change in agitation score. That is, the estimation unit 120 can estimate, based on past case examples, the countermeasure information in accordance with the change in features of the biological information in the predetermined period of time or with the change in agitation score. Specifically, in response to a change in agitation score during a time zone between “17:00:00 on Jul. 11, 2017” and “22:00:00 on Jul. 11, 2017”, the estimation unit 120 can estimate the countermeasure information, for example, at “22:00:00 on Jul. 11, 2017” in accordance with the agitation score during the above-mentioned time zone and the countermeasure prediction parameters. As the countermeasure prediction parameters in this case, those which are learned between, for example, “0:00:00 on Jul. 1, 2017” and “23:59:59 on Jul. 10, 2017” may be used. Thus, the estimation unit 120 can also estimate that the target patient may be shifted into a non-normal state (agitation state) soon although the target patient is in a normal state (non-agitation state) at a time instant of “22:00:00 on Jul. 11, 2017”. In this event, the estimation unit 120 can estimate the countermeasure information including the countermeasure which can prevent the target patient in the normal state (non-agitation state) from being shifted into the non-normal state (agitation state).

The estimation unit 120 may estimate the countermeasure information in consideration of additional information in addition to the discrimination information. In this example embodiment, the additional information means information which exerts an influence upon the discrimination information of the target patient. Specifically, the additional information means, for example, surrounding conditions of the target patient and an influence exerted upon the surroundings by performing the countermeasure (surrounding environment information), a magnitude of a load imposed on the patient (patient load), a magnitude of a load imposed on the nurse or the like (responder load), a time interval required to perform the countermeasure on the target patient, a financial cost required by performing the countermeasure, and information included in a medical chart (electronic medical chart). In this event, the estimation unit 120 can estimate, by considering the additional information, the countermeasure information in consideration of the loads imposed on the target patient and the nurse or the like, the influence exerted upon patients around him/her by performing the countermeasure on the target patient, and so on. That is, since the estimation unit 120 estimates the discrimination information in consideration of the additional information, accuracy of the discrimination information is improved and the loads imposed on the target patient and the nurse or the like are further decreased.

The surrounding environment information is information including a degree of exerting an influence upon the surroundings, such as causing trouble to those patients around the target patient, upon performing the countermeasure on the target patient. Specifically, the surrounding environment information means, for example, information indicating whether or not a sickroom of the target patient is a private room, a distance between the sickroom and a nurses' station, whether or not the target patient must be moved from the room in order to perform the countermeasure, whether or not a time of performing the countermeasure is the daytime, whether or not the room must be lightened upon performing the countermeasure after lights-out, and whether or not a sound is produced upon performing the countermeasure after lights-out. The above-mentioned surrounding environment information is an example and does not limit the present invention.

The patient load means a load which is imposed on a body of the target patient by performing the countermeasure. For example, the load becomes large in the countermeasure of administering a strong sedative drug to the target patient whereas the load becomes small in the countermeasure of calling to the target patient.

The responder load means a load which is imposed on the nurse or the like by performing the countermeasure. For example, the load becomes small in a case of administering an effective sedative drug to the target patient whereas the load becomes large in a case of continuously calling to the target patient.

The information included in the electronic medical chart means information related to, for example, age, sex, height, weight, a family structure, presence or absence of a complicating disease, administration history, blood components, a medical history, elimination, and eating and drinking. The above-mentioned information included in the electronic medical chart is an example and does not limit the present invention.

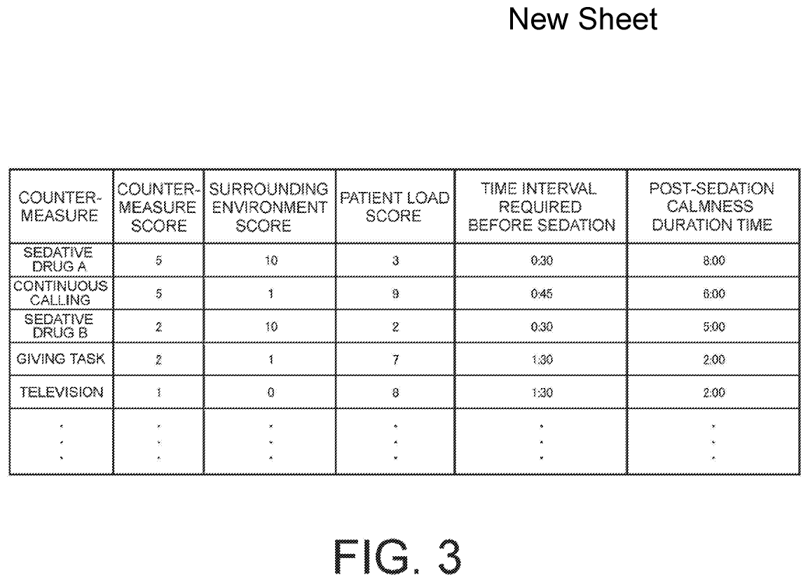

FIG. 3 is a view for illustrating an example of the countermeasure information estimated by the estimation unit 120. As illustrated in FIG. 3, the countermeasure information includes, for example, a countermeasure, a countermeasure score, a surrounding environment score, a patient load score, a time interval required before sedation, and a post-sedation calmness duration time. The surrounding environment score and the patient load score are information related to the additional information whereas the time interval required before the sedation and the post-sedation calmness duration time are information related to the discrimination information. Although the countermeasure information illustrated in FIG. 3 includes, as the additional information, two kinds of information, i.e., the surrounding environment score and the patient load score, this is an example. The countermeasure information may further include a plurality of kinds of additional information or may not include any additional information.

The countermeasures are kinds of procedures to be performed on the target patient and mean information related to, for example, procedures for bringing the target patient into a state where any problem is not caused or procedures for suppressing the non-normal state (agitation state) of the target patient. Specifically, the countermeasures are classified into countermeasures which are mainly performed in nighttime and countermeasures which are mainly performed in daytime. The countermeasures which are mainly performed in nighttime include, for example, taking him/her to a toilet, disposal of excreta, making him/her drink a beverage, administering a sedative drug, directly calling to him/her, adjusting body temperature, restraining his/her body, calling to him/her via a television telephone or the like, making him/her hear music, making him/her smell a scent (aroma), and giving him/her a concentrating task (work). On the other hand, the countermeasures which are mainly performed in daytime include, for example, administering a sedative drug, directly calling to him/her, calling to him/her via a television telephone or the like, making him/her do exercise (undergo rehabilitation), making him/her eat, adjusting a sleeping time zone, adjusting illumination of a room, adjusting body temperature, adjusting temperature of the room, making him/her have a bath, making him/her watch a television program, making him/her hear music, and making him/her smell a scent (aroma). The above-mentioned countermeasures are merely examples and do not limit the present invention. The responder such as the nurse or the like can easily suppress the non-normal state (agitation state) of the target patient by following the countermeasures, for example, while suppressing the loads imposed on the target patient and the nurse or the like and an adverse influence exerted on other patients around him/her by performing the countermeasure for the target patient. Herein, the adverse influence means an influence, for example, that sleeping patients around him/her are woken, the patients around him/her cannot sleep, or the patients around him/her get angry due to noisiness.

Since a movement (temporal change) of the agitation score illustrated in FIG. 2 differs depending on every target patient, the estimation unit 120 can estimate the countermeasure information which is different for every patient even if values of the agitation scores are the same at a particular timing. In addition, the estimation unit 120 may estimate a different countermeasure in accordance with the magnitude of the agitation score. Specifically, the estimation unit 120 can estimate, for example, as a main countermeasure, the countermeasure having a large sedative effect in a case where the agitation score is relatively large and can estimate, as the main countermeasure, the countermeasure with a small load imposed on the body with respect to a physically weak target patient even if the agitation score is large.

The countermeasure score means a value indicating effectiveness of the countermeasure which is performed on the target patient. The countermeasure score is, for example, represented by five levels of 1 to 5 and means that the countermeasure has a greater effect as a numeral thereof is larger. Specifically, the countermeasure information illustrated in FIG. 3 indicates that, for the target patient, administering a pain-relief drug A and continuously calling to him/her have a large effect whereas making him/her watch a television program has a small effect. The countermeasure score may be represented by a greater number of levels than the five levels or may be represented by a smaller number of levels than the five levels. That is, the respective countermeasures included in the countermeasure information of this example embodiment are associated with the countermeasure scores. Thus, in this example embodiment, degrees of the effect and accuracy of the countermeasures included in the countermeasure information become explicit. Therefore, the nurse or the like can easily grasp the effect of the countermeasure by referring to the countermeasure score.

The surrounding environment score means a value indicating an influence exerted on the surrounding environment by performing the countermeasure on the countermeasure patient. The surrounding environment score is, for example, represented by ten levels of 1 to 10 and means that the influence exerted on the surrounding environment is smaller as a numeral thereof is larger. Specifically, the countermeasure information illustrated in FIG. 3 indicates that, for the target patient, administering the pain-relief drug A and administering a pain-relief drug B have a small influence exerted on the surrounding environment whereas making him/her watch a television program has a large influence exerted on the surrounding environment in case of sharing a room because a television emits light and sound. The surrounding environment score may be represented by a greater number of levels than the ten levels or may be represented by a smaller number of levels than the ten levels.

The patient load score means a value indicating a magnitude of a load imposed on the patient by performing the countermeasure on the target patient. The patient load score is a score represented by, for example, ten levels of 1 to 10 and means that the load is smaller as a numeral thereof is larger. Specifically, the countermeasure information illustrated in FIG. 3 indicates that, for the target patient, administering the pain-relief drug A and administering the pain-relief drug B impose a large load on the patient whereas continuous calling imposes a small load on the patient. The patient load score may be represented by a greater number of levels than the ten levels or may be represented by a smaller number of levels than the ten levels.

In a case where the countermeasure information includes, as the additional information, information other than the surrounding environment score and the patient load score, for example, a score may be evaluated with levels of 1 to 10 in the same manner as the surrounding environment score and the patient load score to include the evaluated additional information into the countermeasure information.

The time interval required before the sedation is a predicted time interval required for the state of the target patient to be shifted from the agitation state to the non-agitation state as a result of performing the countermeasure on the target patient. Specifically, FIG. 3 shows that, when the pain-relief drug A is administered to the target patient which is put into the agitation state, the target patient is shifted from the agitation state to the non-agitation state after lapse of thirty minutes from administration of the pain-relief drug A. The transition from the agitation state to the non-agitation state can be determined, for example, based on the fact that the agitation score drops from a value not less than the threshold value to a value less than the threshold value.

The post-sedation calmness duration time is a predicted time interval during which the non-agitation state continues after the state of the target patient is shifted from the agitation state to the non-agitation state. Specifically, FIG. 3 shows that, when the pain-relief drug A is administered to the target patient, the non-agitation state of the target patient continues for eight hours. A duration time of the non-agitation state can be determined, for example, based on a duration time during which the agitation score is less than the threshold value.

[Operation of the Biological Information Processing System 100]

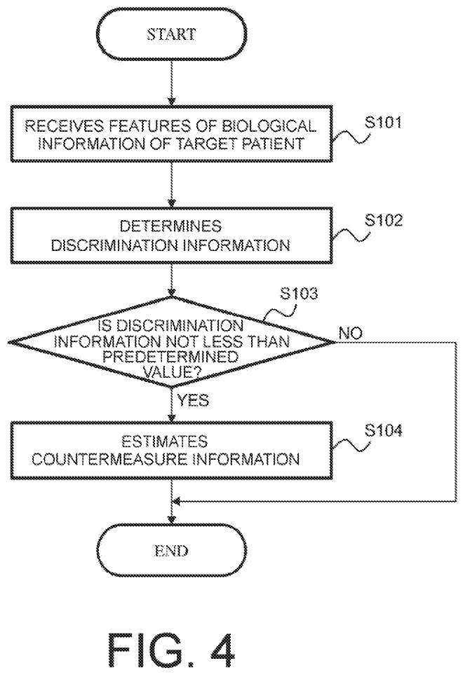

FIG. 4 is a flow chart for illustrating a flow of operation of the biological information processing system 100 illustrated in FIG. 1. Hereinafter, the flow of the operation of the biological information processing system 100 will be described with reference to FIG. 1 and FIG. 4.

First, the determination unit 110 receives features related to biological information of the target patient from the outside (Step S101).

Next, the determination unit 110 determines, based on the features related to the biological information and discrimination parameters, discrimination information indicating whether the target patient is agitated or non-agitated (Step S102).

Next, when the value of the discrimination information (agitation score) is less than a predetermined value (“NO” in Step S103), the biological information processing system 100 terminates its operation. On the other hand, when the value of the discrimination information (agitation score) is not less than the predetermined value (“YES” in Step S103), the estimation unit 120 estimates countermeasure information in accordance with the discrimination information (Step S104).

As described above, the biological information processing system 100 according to this example embodiment can estimate the countermeasure information as illustrated in FIG. 3 based on the past case examples. Therefore, the nurse or the like can perform an optimum countermeasure on the target patient by considering the countermeasure information illustrated in FIG. 3. Accordingly, this example embodiment can reduce the load on the nurse or the like, can prevent an injury of the target patient, and can prevent a delay in treatment caused by performing a less effective countermeasure. In addition, in this example embodiment, the nurse or the like can predict a transition to the agitation state while the target patient is in the non-agitation state, and can perform the countermeasure on the target patient in question. Thus, in this example embodiment, it is possible to prevent the target patient from being shifted from the non-agitation state to the agitation state. Furthermore, the biological information processing system 100 can estimate the countermeasure information also in consideration of the influence on the surrounding environment that is exerted by performing the countermeasure. Therefore, in a case where there are a plurality of countermeasures which are substantially same in effect, the nurse or the like can avoid, for example, a countermeasure which would cause trouble to the patients around the target patient. Accordingly, this example embodiment can suppress the influence on the surrounding environment.

### Second Example Embodiment

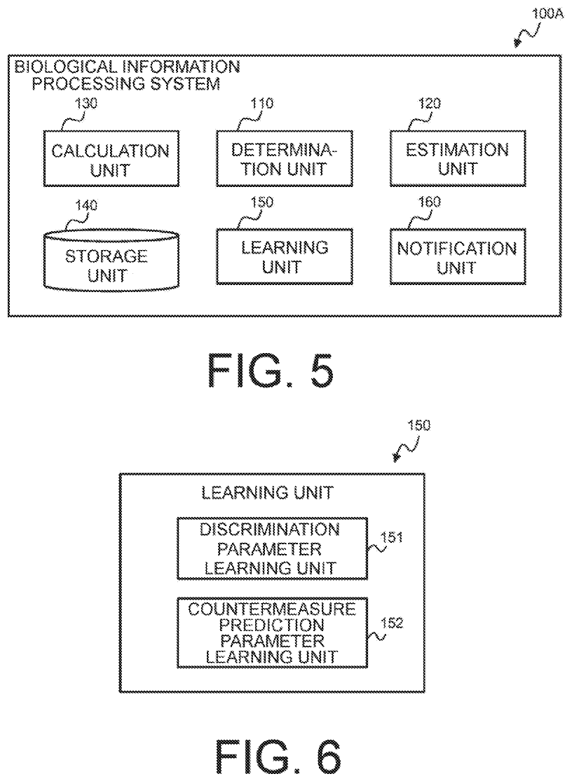

FIG. 5 is a block diagram for illustrating a biological information processing system according to a second example embodiment of the present invention. As illustrated in FIG. 5, the biological information processing system 100A comprises the determination unit 110, the estimation unit 120, a calculation unit 130, a storage unite 140, and a learning unit 150, and a notification unit 160.

The calculation unit 130 receives the biological information of the target patient that is detected by a biological sensor (not shown) or the like and calculates the features related to this biological information. Note that the calculation unit 130 may acquire the features related to the biological information from the outside.

The storage unit 140 at least holds the countermeasure information, the discrimination information, the discrimination parameters, and countermeasure prediction parameters. In this event, the determination unit 110 determines the discrimination information based on the features of the biological information acquired by the calculation unit 130 and the discrimination parameters held in the storage unit 140. The determination unit 110 may have a function of storing the determined discrimination information in the storage unit 140. The estimation unit 120 estimates the countermeasure information based on the discrimination information determined by the determination unit 110 and the countermeasure prediction parameters held in the storage unit 140. In addition, the estimation unit 120 may have a function of storing the estimated countermeasure information in the storage unit 140.

The learning unit 150 can learn, using machine learning, the discrimination parameters and the countermeasure prediction parameters. Specifically, as illustrated in FIG. 6, the learning unit 150 comprises a discrimination parameter learning unit 151 and a countermeasure prediction parameter learning unit 152.

The discrimination parameter learning unit 151 learns the discrimination parameters by learning a relationship between features of a plurality of pieces of biological information in the past and whether the target patient is in the agitation state or the non-agitation state. In addition, the discrimination parameter learning unit 151 can store the generated discrimination parameters in the storage unit 140.

The countermeasure prediction parameter learning unit 152 learns the countermeasure prediction parameters based on a plurality of countermeasures performed when a plurality of patients including the target patient are in the agitation state, respectively, and a plurality of features related to the biological information of the plurality of patients, respectively, in a predetermined time interval. In addition, the countermeasure prediction parameter learning unit 152 can store the generated countermeasure prediction parameters in the storage unit 140. In this example embodiment, the biological information processing system 100A has a function of learning the countermeasure prediction parameters. Therefore, this example embodiment can improve accuracy of the countermeasure information by repeating the learning.

The notification unit 160 notifies the nurse or the like of the countermeasure information estimated by the estimation unit 120. The notification unit 160 is configured to automatically notify of the countermeasure information, for example, via a voice or an image after the estimation unit 120 estimates the countermeasure information. Such a notification unit 160 may be configured by, for example, a general loudspeaker or a general display. Accordingly, the nurse or the like can easily grasp the countermeasure information by notification from the notification unit 160. In addition, the notification unit 160 may notify, of the countermeasure information, a portable terminal or a wearable terminal which is possessed by the nurse or the like and which can communicate with the biological information processing system 100A (notification unit 160). Accordingly, the nurse or the like can confirm the estimated countermeasure information even if he/she is not present at a specific location (e.g. in front of the biological information processing system 100A or the like) because the countermeasure information is notified from the notification unit 160.

[Operation of Learning]

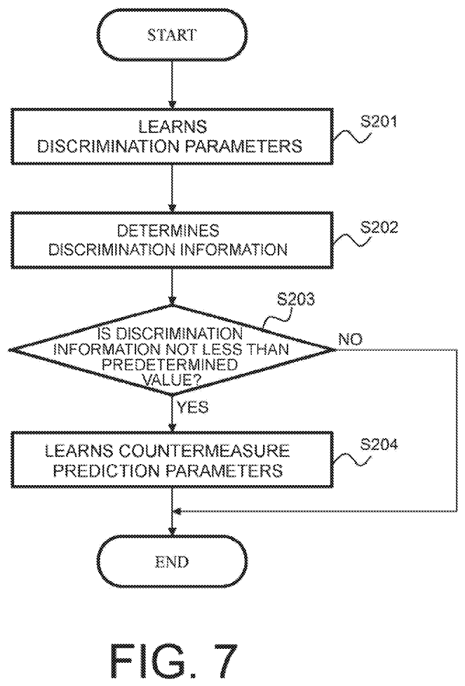

Next referring to FIGS. 5, 6, and 7, description will proceed to a flow of operation of learning the discrimination parameters and the countermeasure prediction parameters by the biological information processing system 100A. FIG. 7 is a flow chart for illustrating a flow of operation when the biological information processing system 100A learns the discrimination parameters and the countermeasure prediction parameters.

First, the discrimination parameter learning unit 151 learns the discrimination parameters (Step S201). Specifically, the discrimination parameter learning unit 151 learns, by machine learning, the discrimination parameters by using, as training data, features calculated from the past biological information of the target patient that has been measured in the agitation state and features calculated from the past biological information of the target patient that has been measured in the non-agitation state.

Next, the determination unit 110 determines, based on the measured biological information of the target patient and the discrimination parameters, the discrimination information indicating whether the target patient is in the “agitation state” or the “non-agitation state” (Step S202).

Next, when the value of the discrimination information is less than a predetermined value in Step S203 (“NO” in Step S203), the biological information processing system 100A terminates the operation of learning because the nurse or the like does not perform the countermeasure on the target patient.

On the other hand, when the value of the discrimination information is not less than the predetermined value in Step S203 (“YES” in Step S203), the countermeasure prediction parameter learning unit 152 learns the countermeasure prediction parameters (Step S204). Specifically, the countermeasure prediction parameter learning unit 152 learns the countermeasure prediction parameters by machine learning a relationship between the countermeasures performed on the target patient and temporal changes of the discrimination information of the target patient as a result of performing the countermeasures.

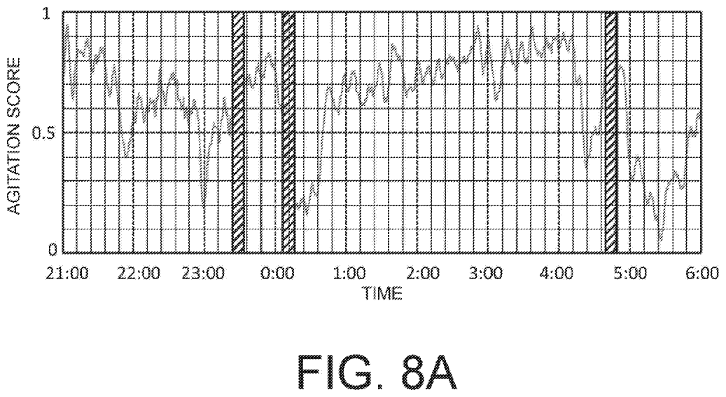

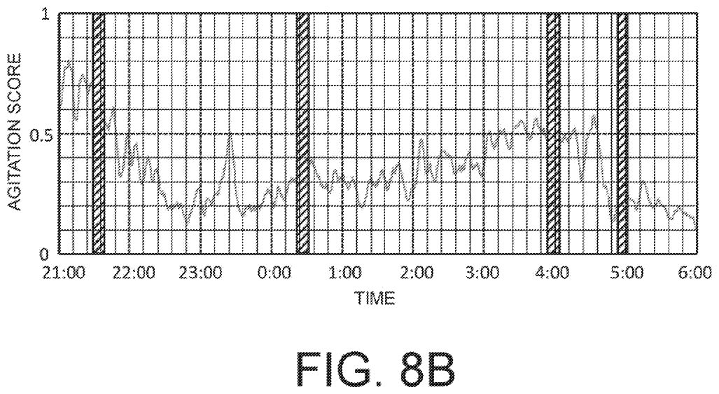

FIGS. 8A and 8B are views for illustrating temporal changes of values (agitation score) of the discrimination information of the target patient, where the axis of abscissas represents a time whereas the axis of ordinate represents the agitation score. In FIGS. 8A and 8B, an area designated by hatched lines means that the nurse or the like performs the countermeasure on the target patient.

Specifically, FIG. 8A illustrates that, although the nurse or the like performs the countermeasure on the target patient at about “0:10”, the agitation score in a midnight time zone is high and the target patient is in the agitation state in the midnight time zone. That is, FIG. 8A serves as training data indicating an example in which the countermeasure has no effect.

On the other hand, FIG. 8B illustrates that, as a result of performing the countermeasure on the target patient by the nurse or the like at about “0:20”, the agitation score of the midnight time zone is kept low and the target patient is in the non-agitation state in the midnight time zone. That is, FIG. 8B serves as training data indicating an example in which the countermeasure has an effect.

The countermeasure prediction parameter learning unit 152 learns the countermeasure prediction parameters by using a lot of training data as illustrated in FIGS. 8A and 8B. Since the countermeasure prediction parameter learning unit 152 uses the machine learning, accuracy of the countermeasure prediction parameters is improved as an amount of learned data increases.

[Operation of Biological Information Processing System 100A]

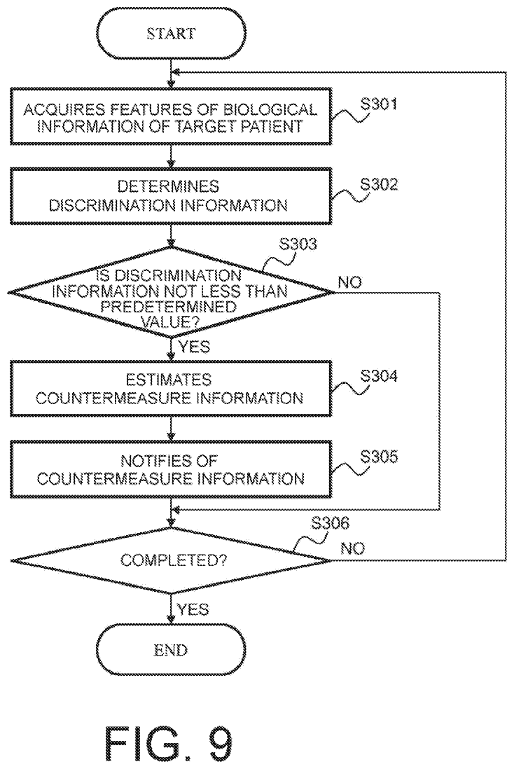

FIG. 9 is a flow chart for illustrating a flow of operation until the biological information processing system 100A illustrated in FIG. 5 notifies of the countermeasure information after acquiring the biological information of the target patient. Now, the flow of the operation of the biological information processing system 100A will be described with reference to FIG. 5 and FIG. 9.

First, the calculation unit 130 receives the biological information of the target patient that is measured by the biological sensor or the like and calculates the features related to the biological information (Step S301). At this time, the calculation unit 130 may acquire the features related to the biological information of the target patient from the outside.

Next, the determination unit 110 determines, based on the features calculated by the calculation unit 130 and the discrimination parameters held in the storage unit 140, the discrimination information indicating whether the target patient is in the “agitation state” or the “non-agitation state” (Step S302).

Next, when a value of the discrimination information is not less than a predetermined value (“YES” in Step S303), the estimation unit 120 estimates, based on the discrimination information and the countermeasure prediction parameters held in the storage unit 140, the countermeasure information including at least one countermeasure to be performed on the target patient (Step S304).

Next, the notification unit 160 notifies the nurse or the like of the countermeasure information estimated by the estimation unit 120 (Step S305).

Then, after Step S305 or when the value of the discrimination information is less than the predetermined value in Step S303 (“NO” in Step S303), the biological information processing system 100A terminates the processing (“YES” in Step S306) if a necessity of detection of continuous agitation states of the target patient is removed or the target patient leaves a hospital. On the other hand, if the necessity of detection of continuous agitation states of the target patient is not removed or the target patient continuously stays in the hospital (“NO” in Step S306), the biological information processing system 100A is turned back to Step S301.

[Hardware Configuration of Biological Information Processing System]

The biological information processing system 100 and the biological information processing system 100A mentioned above may be implemented by hardware or may be implemented by software. In addition, the biological information processing system 100 and the biological information processing system 100A may be implemented by a combination of hardware and software.

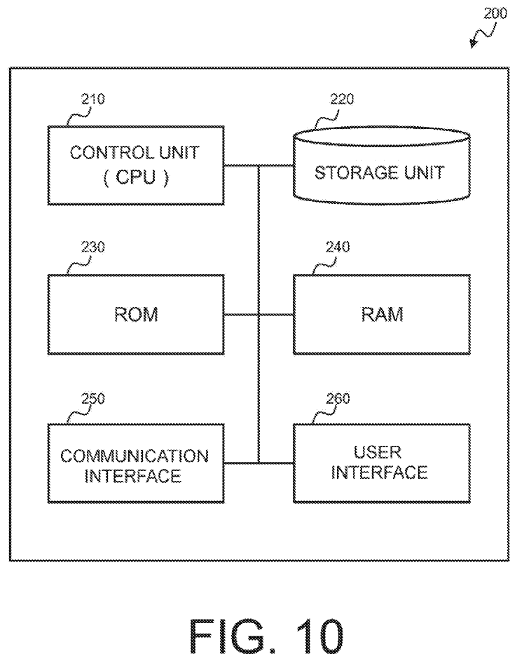

FIG. 10 is a block diagram for illustrating one example of an information processing apparatus (computer) constituting the biological information processing system 100 and the biological information processing system 100A.

As shown in FIG. 10, the information processing apparatus 200 comprises a control unit (CPU: Central Processing Unit) 210, a storage unit 220, an ROM (Read Only Memory) 230, an RAM (Random Access Memory) 240, a communication interface 250, and a user interface 260.

The control unit (CPU) 210 may implement various functions of the biological information processing system 100 and the biological information processing system 100A by developing and executing, in the RAM 240, a program which is stored in the storage unit 220 or the ROM 230. In addition, the control unit (CPU) 210 may comprise an internal buffer which is adapted to temporarily store data or the like.

The storage unit 220 comprises a bulk storage medium which can hold various types of data and may be implemented by a storage medium such as an HDD (Hard Disk Drive) and an SSD (Solid State Drive). The storage unit 220 may be a cloud storage existing in a communication network when the information processing apparatus 200 is connected to the communication network via the communication interface 250. The storage unit 220 may hold a program readable by the control unit (CPU) 210.

The ROM 230 is a nonvolatile storage device which may comprise a flash memory having a small capacity as compared to the storage unit 220. The ROM 230 may hold a program which is readable by the control unit (CPU) 210. The program readable by the control unit (CPU) 210 may be held in at least one of the storage unit 220 and the ROM 230.

The program readable by the control unit (CPU) 210 may be supplied to the information processing apparatus 200 in a state where it is non-transitorily stored in various storage media readable by the computer. Such storage media include, for example, a magnetic tape, a magnetic disk, a magneto-optical disc, a CD-ROM (Compact Disc-Read Only Memory), a CD-R (Compact Disc-Readable), a CD-RW (Compact Disc-ReWritable), and a semiconductor memory.

The RAM 240 comprises a semiconductor memory such as a DRAM (Dynamic Random Access Memory) and an SRAM (Static Random Access Memory) and may be used as an internal buffer which temporarily stores data and so on.

The communication interface 250 is an interface which connects the information processing system 200 and the communication network via wire or wirelessly.

The user interface 260 comprises, for example, a displaying unit such as a display and an input unit such as a keyboard, a mouse, and a touch panel.

While the present invention has been described with reference to the example embodiments thereof, the present invention is not limited thereto. For example, the present invention encompasses configurations obtained by appropriately combining parts or a whole of the example embodiments described so far as well as configurations obtained by appropriately modifying the above-mentioned configurations.

A part or a whole of the example embodiments described above may also be described as the following supplementary notes without being limited thereto.

(Supplementary Note 1)

A biological information processing system comprising:

a determination unit configured to determine, based on features of input biological information of a target patient, discrimination information indicating whether or not a condition of the target patient has changed in comparison with a normal state; and

an estimation unit configured to estimate countermeasure information for the target patient based on the discrimination information and countermeasure prediction parameters which are preliminary learned.

(Supplementary Note 2)

The biological information processing system according to Supplementary Note 1, further comprising:

a learning unit configured to learn the countermeasure prediction parameters based on a plurality of countermeasures to be performed when a plurality of patients are in non-normal states, respectively, and a plurality of features related to respective biological information of the plurality of patients in a predetermined period of time; and

a storage unit configure to hold the learned countermeasure prediction parameters.

(Supplementary Note 3)

The biological information processing system according to Supplementary Note 2, wherein the estimation unit is configured to estimate the countermeasure information with the plurality of the countermeasures associated with countermeasure scores, respectively.

(Supplementary Note 4)

The biological information processing system according to any one of Supplementary Notes 1 to 3, wherein the estimation unit is configured to estimate the countermeasure information in consideration of additional information related to the target patient.

(Supplementary Note 5)

The biological information processing system according to any one of Supplementary Notes 1 to 4, further comprising a notification unit configured to notify a user of the estimated countermeasure information.

(Supplementary Note 6)

The biological information processing system according to any one of Supplementary Notes 1 to 5, wherein the determination unit is configured to determine the discrimination information based on discrimination parameters which are preliminarily learned and the features related to the biological information of the target patient.

(Supplementary Note 7)

The biological information processing system according to any one of Supplementary Notes 1 to 6, wherein the discrimination information includes an agitation score correlated with a possibility of a non-normal state.

(Supplementary Note 8)

A biological information processing method comprising:

determining, by a determination unit, based on features of input biological information of a target patient, discrimination information indicating whether or not a condition of the target patient has changed in comparison with a normal state; and

estimating, by an estimation unit, countermeasure information for the target patient based on the discrimination information and countermeasure prediction parameters which are preliminarily learned.

(Supplementary Note 9)

The biological information processing method according to Supplementary Note 8, comprising:

learning, by a learning unit, the countermeasure prediction parameters based on a plurality of countermeasures to be performed when a plurality of patients are in non-normal states, respectively, and a plurality of features related to respective biological information of the plurality of patients in a predetermined period of time; and

storing the learned countermeasure prediction parameters in a storage unit.

(Supplementary Note 10)

The biological information processing method according to Supplementary Note 9, wherein:

the estimation unit estimates the countermeasure information with the plurality of the countermeasures associated with countermeasure scores, respectively.

(Supplementary Note 11)

The biological information processing method according to any one of Supplementary Notes 8 to 10, wherein the estimation unit estimates the countermeasure information in consideration of additional information related to the target patient.

(Supplementary Note 12)

The biological information processing method according to any one of Supplementary Notes 8 to 11, comprising notifying, by a notification unit, a user of the estimated countermeasure information.

(Supplementary Note 13)

The biological information processing method according to any one of Supplementary Notes 8 to 12, wherein the determination unit determines the discrimination information based on discrimination parameters which are preliminarily learned and the features related to the biological information of the target patient.

(Supplementary Note 14)

A recording medium recording a biological information processing program which causes a computer to execute the processes of:

determining, based on features of input biological information of a target patient, discrimination information indicating whether or not a condition of the target patient has changed in comparison with a normal state; and

estimating countermeasure information for the target patient based on the identification information and countermeasure prediction parameters which are preliminarily learned.

(Supplementary Note 15)

The biological information processing program recording medium according to Supplementary Note 14, wherein the biological information processing program causes the computer to further execute the processes of:

learning the countermeasure prediction parameters based on a plurality of countermeasures to be performed when a plurality of patients are in non-normal states, respectively, and a plurality of features related to respective biological information of the plurality of patients in a predetermined period of time; and

storing the learned treatment prediction parameters in a storage unit.

(Supplementary Note 16)

The biological information processing program recording medium according to Supplementary Note 15, wherein the biological information processing program causes the computer to execute the process of:

estimating the countermeasure information with the plurality of the countermeasures associated with countermeasure scores, respectively.

(Supplementary Note 17)

The biological information processing program recording medium according to any one of Supplementary Notes 14 to 16, wherein the biological information processing program causes the computer to execute the process of:

estimating the countermeasure information in consideration of additional information related to the target patient.

(Supplementary Note 18)

The biological information processing program recording medium according to any one of Supplementary Notes 14 to 17, wherein the biological information processing program causes the computer to further execute the process of:
notifying a user of the estimated countermeasure information.

(Supplementary Note 19)

The biological information processing program recording medium according to any one of Supplementary Notes 14 to 18, wherein the biological information processing program causes the computer to execute the process of:

determining the discrimination information based on discrimination parameters which are preliminarily learned and the features related to the biological information of the target patient.

This application is based upon and claims the benefit of priority from Japanese patent application No. 2017-196797, filed on Oct. 10, 2017, the disclosure of which is incorporated herein in its entirety by reference.

### REFERENCE SIGNS LIST

100, 100A biological information processing system

110 determination unit

120 estimation unit

130 calculation unit

140 storage unit

150 learning unit

151 discrimination parameter learning unit

152 countermeasure prediction parameter learning unit

160 notification unit

200 information processing apparatus

210 control unit (CPU)

220 storage unit

230 ROM

240 RAM

250 communication interface

260 user interface

1. A biological information processing system comprising:
a determination unit configured to determine, based on features of input biological information of a target patient, discrimination information indicating whether or not a condition of the target patient has changed in comparison with a normal state; and
an estimation unit configured to estimate countermeasure information for the target patient based on the discrimination information and countermeasure prediction parameters which are preliminary learned.

2. The biological information processing system as claimed in claim 1, further comprising:
a learning unit configured to learn the countermeasure prediction parameters based on a plurality of countermeasures and a plurality of features and
a storage unit configure to hold the learned countermeasure prediction parameters.

3. The biological information processing system as claimed in claim 2, wherein the estimation unit is configured to estimate the countermeasure information with the plurality of the countermeasures associated with countermeasure scores, respectively.

4. The biological information processing system as claimed in claim 1, wherein the estimation unit is configured to estimate the countermeasure information in consideration of additional information related to the target patient.

5. The biological information processing system as claimed in claim 1, further comprising a notification unit configured to notify a user of the estimated countermeasure information.

6. The biological information processing system as claimed in claim 1, further comprising a notification unit configured to notify a user of the estimated countermeasure information.

7. The biological information processing system as claimed in claim 1, wherein the discrimination information includes an agitation score correlated with a possibility of a non-normal state.

8. A biological information processing method comprising:
determining, based on features of input biological information of a target patient, discrimination information indicating whether or not a condition of the target patient has changed in comparison with a normal state; and estimating, countermeasure information for the target patient based on the discrimination information and countermeasure prediction parameters which are preliminarily learned.

9. The biological information processing method as claimed in claim 8, comprising:
learning the countermeasure prediction parameters based on a plurality of countermeasures to be performed when a plurality of patients are in non-normal states, respectively, and a plurality of features related to respective biological information of the plurality of patients in a predetermined period of time; and
storing the learned countermeasure prediction parameters in a storage unit.

10. The biological information processing method as claimed in claim 9, wherein:
the estimating estimates the countermeasure information with the plurality of the countermeasures associated with countermeasure scores, respectively.

11. The biological information processing method as claimed in claim 8, wherein the estimating estimates the countermeasure information in consideration of additional information related to the target patient.

12. The biological information processing method as claimed in claim 8, comprising notifying a user of the estimated countermeasure information.

13. The biological information processing method as claimed in claim 8, wherein the determining determines the discrimination information based on discrimination parameters which are preliminarily learned and the features related to the biological information of the target patient.

14. A non-transitory recording medium recording a biological information processing program which causes a computer to execute the processes of:
determining, based on features of input biological information of a target patient, discrimination information indicating whether or not a condition of the target patient has changed in comparison with a normal state; and
estimating countermeasure information for the target patient based on the identification information and countermeasure prediction parameters which are preliminarily learned.

15. The non-transitory recording medium as claimed in claim 14, wherein the biological information processing program causes the computer to further execute the processes of:
learning the countermeasure prediction parameters based on a plurality of countermeasures to be performed when a plurality of patients are in non-normal states, respectively, and a plurality of features related to respective biological information of the plurality of patients in a predetermined period of time; and
storing the learned treatment prediction parameters in a storage unit.

16. The non-transitory recording medium as claimed in claim 15, wherein the biological information processing program causes the computer to execute the process of:
estimating the countermeasure information with the plurality of the countermeasures associated with countermeasure scores, respectively.

17. The non-transitory biological recording medium as claimed in claim 14, wherein the biological information processing program causes the computer to execute the process of:
estimating the countermeasure information in consideration of additional information related to the target patient.

18. The non-transitory recording medium as claimed in claim 14, wherein the biological information processing program causes the computer to further execute the process of:
notifying a user of the estimated countermeasure information.

19. The non-transitory recording medium as claimed in claim 14, wherein the biological information processing program causes the computer to execute the process of:
determining the discrimination information based on discrimination parameters which are preliminarily learned and the features related to the biological information of the target patient.
